(19) United States
(12) Patent Application Publication
(10) Pub. No.: US 2016/0324370 A1
(43) Pub. Date: Nov. 10, 2016

(54) AN IMPROVED IN-SHELL EGG SCRAMBLER

(71) Applicant: EGGLOGIX LTD, Tel Aviv (IL)

(72) Inventor: ASSAF GURNER, Tel Aviv (IL)

(21) Appl. No.: 15/034,516

(22) PCT Filed: Nov. 3, 2014
(86) PCT No.: PCT/IL2014/050951

§ 371 Date: May 4, 2016

Related U.S. Application Data
(60) Provisional application No. 61/903,906, filed on Nov. 13, 2013.

(51) Int. Cl. A47J 43/10; B01F 9/10; B01F 15/00; G01N 33/08

(52) U.S. Cl. CPC A47J 43/1006; G01N 33/085; B01F 9/10; B01F 15/00389; B01F 15/00207

(57) ABSTRACT

The present invention disclosed additional improved embodiments of an in-shell-egg-scrambler. In-shell-egg-scrambler was disclosed previously in some publications and the present invention provides some improved embodiments of the scrambler.

The present invention relates to an in-shell-egg-scrambler that was disclosed in U.S. Pat. No. 8,265,166, in WO2008/139450 and in a PCT application PCT/IL2013/050599.

### FIELD OF THE INVENTION

### Background of the Invention

An in-shell-egg-scrambler was previously described in details in the above mentioned publications. The present invention provides the same in-shell-egg-scrambler with some significant improvements and provides a manually operated embodiment.

### SUMMARY OF THE INVENTION

The present invention provides an improved in-shell-egg-scrambler. The present invention provides additional improved embodiments of the in-shell-egg-scrambler that was disclosed in the previous publications.

According to the principles of the present invention an improved in-shell egg scrambler is provided.

According to a preferred embodiment of the present invention the scramble is comprised of a rotation unit operatively connected to an egg—placed perpendicularly along the long axis of the egg—retention portion designed to rotate the egg retention portion such that an egg retained therein is scrambled without breaking the shell thereof, wherein the rotation unit spins the egg retention portion, with the egg retained therein, by repeatedly accelerating the rotation from static to a predetermined velocity then stopping, for a predetermined time in the same direction or until the retained egg is scrambled and wherein the rotations are a series of rotation pulses, wherein the rise time of each pulse is less than 100 milliseconds, each pulse duration is less than a second and wherein the whole process is less than a minute, in order to prevent the diffusion/penetration of air from the air egg cell into the egg's liquid and when using low torque the rotational pulses are less than 3 seconds, and wherein the whole process is less than 2 minutes.

According to another preferred embodiment the scrambler is further includes a controller and a sensor for recognizing that the retained egg is scrambled operative for set the pulses' parameters according to the information received from the sensor and to a predetermined criteria.

According to another preferred embodiment the scrambler is provided wherein the sensor is comprised of an illumination source located near the egg's shell and a light sensor located near the next side egg's shell, sensing the transparency change of the egg or the light color changes of the light, which is transmitted through the egg from the illumination source.

According to another preferred embodiment the scrambler is provided, wherein the transparency change or light color changes are recognized visually by the user.

According to another preferred embodiment the scrambler is provided, wherein the controller is operative for activating the rotation unit according to a predetermined routine and stops the rotation unit when the sensor recognizes that the egg is scrambled.

According to another preferred embodiment the scrambler is provided, wherein the sensor detects illumination that illuminated through the egg and compare the detection's parameters with an empiric information.

According to another preferred embodiment the scrambler is provided, wherein the sensor uses any sensing technology such IR, ultrasound, laser, imaging processing or any other sensing technology.

According to another preferred embodiment the scrambler is provided, wherein the rotation unit spins the egg retention portion at a rate which is between 1500-4500 rpm and rapidly stops the rotation unit.

According to another preferred embodiment the scrambler is, further includes an air cell sensor for recognizing that the air of the egg's air cell was released from the cell and reports to the controller.

According to another preferred embodiment the scrambler is provided, wherein the sensor is comprised of a transmitter and receiver that receives—through the egg—the transmission, located near to the highest edge of the egg depending on the egg's position, in order to recognize receiving changes that occurs when the air of the air cell is released and floats up.

According to another preferred embodiment the scrambler is provided, wherein the sensor recognizes the existence of the air cell in its' original location.

According to another preferred embodiment the scrambler is provided, wherein the rotation unit is a container in which the egg can be placed for processing.

According to another preferred embodiment the scrambler is provided, wherein the top edge of the container is held, but not connected, by a pin in order to reduce disordered vibrations.

According to another preferred embodiment the scrambler is provided, wherein the container is held from aside by springy wheels in order to reduce disordered vibrations.

According to another preferred embodiment the scrambler is provided, wherein the container is surrounded by at least one elastic belt in order to reduce disordered vibrations.

According to another aspect of the present invention a manually operated in-shell egg scrambler is provided. This scrambler is comprised of: a housing with top cover and top cover bushing, the bushing capable to hold a shaft; a main shaft with accelerating wheel installed perpendicularly in the housing; a container with upper shaft designed to contain a poultry egg—placed perpendicularly along the long axis of the egg—wherein the bottom of the container is connected to the main shaft and the upper shaft is held by the top cover bushing and wherein the container includes a flexible upper egg holder and flexible lower egg holder; a pulling string coiled around the main shaft and the free edge of the string is protruded from the housing enabling to pull fast the string, which caused the main shaft with the container to rotate—clockwise or anticlockwise—and inertial coiled back the string in the opposite direction; and a string support wheel installed on the main shaft below the coiled string designed to support the string while is coiling back.

According to a preferred embodiment of the manually operated scrambler, the manually scrambler is further includes a light source, wherein the light source located near the egg's shell enables candling the egg from other side in order to observe color or shape changes of the egg's inner to recognize scramble.

### BRIEF DESCRIPTION OF THE DRAWINGS

Various other features and attendant advantages of the present invention will be more fully appreciated from the following detailed description when considered in connection with the accompanying drawings in which like reference characters designate like or corresponding parts throughout the several views, and wherein:

FIG. 1 illustrates a cross section of a preferred embodiment of the scrambler.

FIG. 2 illustrates a cross section of a manually operated scrambler.

### DETAILED DESCRIPTION OF THE ILLUSTRATED EMBODIMENTS

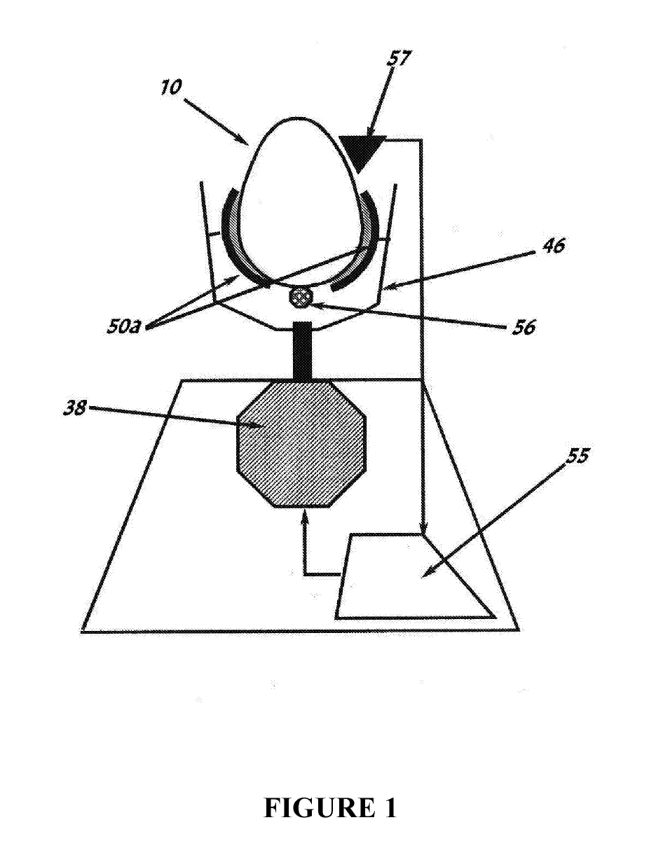

FIG. 1 illustrates a cross section of a preferred embodiment of the scrambler, according to the present invention, in which the egg retention portion comprises a chuck with a plurality of adjustable jaws. The scrambler includes a controller and a sensor with a light source.

In this illustrated embodiment, the egg retention component (46) is a chuck with several adjustable jaws (50a) capable of being adjusted to hold any size egg (10) therewithin or therebetween. Moreover, this embodiment has a controller (55) that controls the rotating unit (38). The controller (55) can be programmed to rotate the retention unit (46) according to direction, velocity, and/or duration, and/or to stop the retention unit (46) when the sensor (57) recognizes that the egg (10) has been scrambled. The sensor (57) recognizes the fact that the egg (10) is scrambled according to light from a light source (56) that is transmitted through the egg (10). When the egg (10) is scrambled, its transparency is changed and the color of the transmitted light is changed.

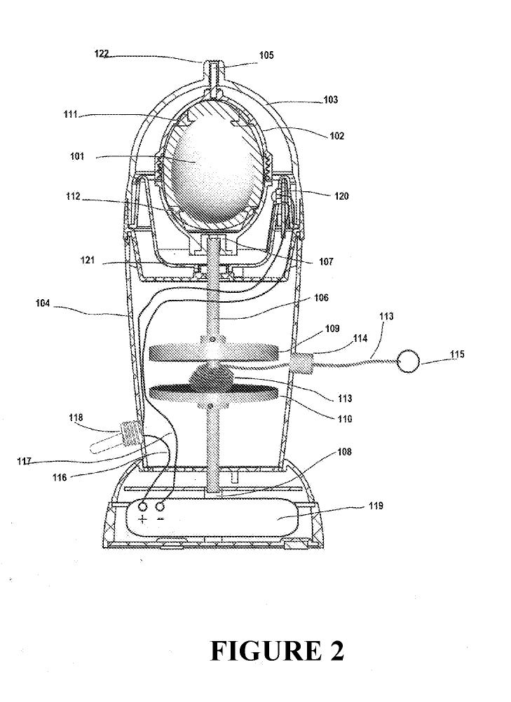

FIG. 2 illustrates the manually operated In-Shell-Egg-Scrambler. The manual scrambler uses two-directional rotation, with high torque generated by a pulling string repeatedly. The main shaft (116) includes acceleration wheel (109) and string support wheel (110), which are tightened to the main shaft (106), which is being inserted into and centralized by the upper bushing (121) and the lower bushing (108). The pull string (113) is connected to the acceleration wheel (109), and goes out of the body through the string leader (114), and is connected to the pulling ring (115). A battery compartment (119) is connected by the electrical wire (116), through the on/off light switch (118), and the return electrical wire (117) to the light source (120). The egg container (102) is mounted on the rubber hex (107) that is attached to the main shaft (106). The top cover (103) is placed on the device while the egg container upper shaft (105), is inserted into the top cover bushing (122). The shell egg (101) is placed in the container (102), and is held centralized between the flexible upper egg holder (111) and the flexible lower egg holder (112). When the container (102) is getting close, the shell egg (101) is held tight between the flexible upper egg holder (111) and the flexible lower egg holder (112). The On/off switch (118) is turned on for activating the light source (120). The light source (120) illuminates the shell egg (101) through the egg shell (candling). The pull string (113) is coiled around the main shaft (106), clockwise, or counter clockwise. The pull string (113) is pulled manually by pulling the pulling ring (115), and causes the main shaft (106) to spin, e.g clockwise, while generating a torque and causes the pull string (113) to uncoil around the main shaft (106). On the same acceleration momentum, and empowered by the acceleration wheel (109), the pull string (113) is recoiled around the main shaft (106) to the counter direction and is getting pulled back into the device, until the pulling ring (115) reaches the body (104) and then the spinning stops, or until the user pulls the pull string (113) ones again for spinning the main shaft (106) to the counter direction, and vice versa. Along the spinning (CW or CCW), the user can see the mixing process evolving, by movements of bright colors and dark colors through the illuminated (candled) shell. When the color seen through the shell looks homogeneously dark color, the egg (101) is mixed in its shell so the user can decide when to stop the process.

1. An in-shell egg scrambler, comprising a rotation unit operatively connected to an egg—placed perpendicularly along the long axis of said egg—retention portion designed to rotate said egg retention portion such that an egg retained therein is scrambled without breaking the shell thereof, wherein said rotation unit spins said egg retention portion, with said egg retained therein, by repeatedly accelerating the rotation from static to a predetermined velocity then stopping, for a predetermined time in the same direction or until said retained egg is scrambled and wherein said rotations are a series of rotation pulses, wherein the rise time of each pulse is less than 100 milliseconds, each pulse duration is less than a second and wherein the whole process is less than a minute, in order to prevent the diffusion/penetration of air from the air egg cell into the egg's liquid and when using low torque said rotational pulses are less than 3 seconds, and wherein the whole process is less than 2 minutes.

2. The in-shell egg scrambler of claim 1, further includes a controller and a sensor for recognizing that said retained egg is scrambled operative for set the pulses' parameters according to the information received from said sensor and to a predetermined criteria.

3. The in-shell egg scrambler of claim 2, wherein said sensor is comprised of an illumination source located near said egg's shell and a light sensor located near the next side egg's shell, sensing the transparency change of said egg or the light color changes of the light, which is transmitted through said egg from said illumination source.

4. The in-shell egg scrambler of claim 3, wherein said transparency change or light color changes are recognized visually by the user.

5. The in-shell egg scrambler of claim 2, wherein said controller is operative for activating said rotation unit according to a predetermined routine and stops said rotation unit when said sensor recognizes that said egg is scrambled.

6. The in-shell egg scrambler of claim 2, wherein said sensor detects illumination that illuminated through said egg and compare said detection's parameters with an empiric information.

7. The in-shell egg scrambler of claim 2, wherein said sensor uses any sensing technology such IR, ultrasound, laser, imaging processing or any other sensing technology.

8. The in-shell egg scrambler of claim 1, wherein said rotation unit spins said egg retention portion at a rate which is between 1500-4500 rpm and rapidly stops said rotation unit.

9. The in-shell egg scrambler of claim 1, further includes an air cell sensor for recognizing that the air of the egg's air cell was released from the cell and reports to said controller.

10. The in-shell egg scrambler of claim 9, wherein said sensor is comprised of a transmitter and receiver that receives—through the egg—said transmission, located near to the highest edge of said egg depending to said egg's position, in order to recognize receiving changes that occurs when the air of said air cell is released and floats up.

11. The in-shell egg scrambler of claim 9, wherein said sensor recognizes the existence of said air cell in its' original location.

12. The in-shell egg scrambler of claim 1, wherein said rotation unit is a container in which the egg can be placed for processing.

13. The in-shell egg scrambler of claim 12, wherein the top edge of said container is held, but not connected, by a pin in order to reduce disordered vibrations.

14. The in-shell egg scrambler of claim 12, wherein said container is held from aside by springy wheels in order to reduce disordered vibrations.

15. The in-shell egg scrambler of claim 12, wherein said container is surrounded by at least one elastic belt in order to reduce disordered vibrations.

16. A manually operated in-shell egg scrambler, said scrambler is comprised of:
a) a housing with top cover and top cover bushing, said bushing capable to hold a shaft;
b) a main shaft with accelerating wheel installed perpendicularly in said housing;
c) a container with upper shaft designed to contain a poultry egg—placed perpendicularly along the long axis of said egg—wherein the bottom of said container is connected to said main shaft and said upper shaft is held by said top cover bushing and wherein said container includes a flexible upper egg holder and flexible lower egg holder;
d) a pulling string coiled around said main shaft and the free edge of said string is protruded from said housing enabling to pull fast said string, which caused said main shaft with said container to rotate—clockwise or anticlockwise—and inertial coiled back said string in the opposite direction; and
e) a string support wheel installed on said main shaft below said coiled string designed to support said string while is coiling back.

17. The manually operated in-shell egg scrambler of claim 16, further includes a light source, wherein said light source located near said egg's shell enables candling said egg from the other side in order to observe color or shape changes of the egg's inner to recognize scramble.

18. The manually operated in-shell egg scrambler of claim 16, wherein said container is designed to be the accelerating wheel.
